(19) United States
(12) Patent Application Publication
(10) Pub. No.: US 2012/0258051 A1
(43) Pub. Date: Oct. 11, 2012

(54) Multistrata nanoparticles and methods for making multistrata nanoparticles

(76) Inventors: Charleson S. Bell, Pawleys Island, SC (US); Shann Yu, Plano, TX (US)

(21) Appl. No.: 13/317,756

(22) Filed: Oct. 27, 2011

Related U.S. Application Data
(60) Provisional application No. 61/473,494, filed on Apr. 8, 2011.

(51) Int. Cl. A61K 49/18; B05D 1/36; A61K 49/04; B82Y 5/00

(52) U.S. Cl. 424/9.42; 424/9.32; 427/2.12; 977/773; 977/927

(57) ABSTRACT

A composition comprising a core comprising an iron oxide, a first shell comprising at least one plasmon active metal at least partially surrounding the core, a second shell comprising a dielectric material at least partially surrounding the first shell, and a third shell comprising at least one plasmon active metal at least partially surrounding the second shell.

### CROSS-REFERENCE TO RELATED APPLICATIONS

This patent application claims priority to U.S. Provisional Patent Application No. 61/473,494 filed Apr. 8, 2011, the entire content of which is incorporated herein by reference.

### STATEMENT REGARDING FEDERAL FUNDING

This invention was made with government support under grant nos. CDMRP #W81XWH-08-1-0502 and IDEAS #W81XWH-05-1-0306 awarded by the Department of Defense. The government has certain rights in the invention.

### INTRODUCTION

Emerging materials and methods in biomedical imaging and biophotonics are improving patient outcomes. More specifically, the utilization of biomedical diagnostics and therapeutic advances in methods such as Magnetic Resonance Imaging (MRI), Computed Tomography (CT) imaging, Photoacoustic Tomography (PAT), Photothermal Optical Coherence Tomography (PT-OCT) and targeted Photothermal Therapy (PTT) have been shown to effectively detect and decrease pathological effects in head-neck cancer, colorectal cancer, and breast cancer.

### SUMMARY

This disclosure provides compositions including a nanoparticle comprising a core comprising iron oxide (e.g., an iron oxide comprising a superparamagnetic iron oxide, such as Fe₂O₃, Fe₃O₄, etc.) a first shell comprising at least one plasmon active metal (e.g., gold, silver, copper, platinum, etc.) at least partially surrounding the core, a second shell comprising a dielectric material (e.g., SiO₂, among others) at least partially surrounding the first shell, and a third shell comprising at least one plasmon active metal (e.g., gold, silver, copper, platinum, etc.) at least partially surrounding the second shell. In some embodiments, the nanoparticle has a diameter less than about 60 nm.

This disclosure also provides methods of making nanoparticles, comprising forming a first shell at least partially surrounding a particle comprising iron oxide (e.g., an iron oxide comprising a superparamagnetic iron oxide, such as Fe₂O₃, Fe₃O₄, etc.), the first shell comprising at least one plasmon active metal (e.g., gold, silver, copper, platinum, etc.), forming a second shell at least partially surrounding the first shell, the second shell comprising a dielectric material (e.g., SiO₂, among others), forming a third shell at least partially surrounding the second shell, the third shell comprising at least one plasmon active metal (e.g., gold, silver, copper, platinum, etc.). The step of forming the first shell may comprise coating the particle with an aminosilane (e.g., APTES, APTMS, APDEMS, APEMS, etc.) to form an aminated core, and coating the aminated core with the first shell. The step of forming the second shell may comprise coating the first shell with the dielectric material using sonication. The step of forming the third shell may comprise coating the second shell with an aminosilane (e.g., APTES, APTMS, APDEMS, or APEMS, or a cyclic aminosilane such as N-n-butyl-aza-2,2-dimethoxysilacyclopentane, etc.) to form an aminated second shell, and coating the aminated second shell with the third shell. In some embodiments, the third shell has an exterior surface with a diameter less than about 60 nm.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic showing an exemplary multistrata nanoparticle (MSNP), and an exemplary method for fabricating the nanoparticle, according to aspects of this disclosure.

FIG. 2 is a series of TEM images of exemplary nanoparticles at various stages of fabrication of MSNPs according to aspects of this disclosure, where: (i) shows the iron-oxide (FeOx) nanoparticles of radius r₁=6 nm, where the scale bar is 100 nm; (ii) shows FeOx-Au nanoparticles (r₁=6, r₂=10±2 nm), where the scale bar is 5 nm; (iv) shows FeOx-Au—SiO₂ nanoparticles (r₁=6, r₂=6.45±0.2, r₃=7.2±0.3 nm) decorated by surrounding Au Duff colloid of 2-5 nm radius, where the scale bar is 1 nm; and (v) shows FeOx-Au—SiO₂—Au MSNPs (r₁=6, r₂=6.45±0.2, r₃=8.3±0.5, r₄=21±5 nm), where the scale bar is 20 nm.

FIG. 3 is plot showing a series of UV-Vis-NIR spectra of exemplary nanoparticles at various stages of fabrication of MSNPs according to this disclosure, where: (i) is the spectra for FeOx nanoparticles of radius r₁=6 nm; (ii) is the spectra for FeOx-Au nanoparticles (r₁=6, r₂=10±2 nm); (iii) is the spectra for FeOx-Au—SiO₂ nanoparticles (r₁=6, r₂=6.45±0.2, r₃=7.2±0.3 nm); (iv) is the spectra for FeOx-Au—SiO₂ nanoparticles (r₁=6, r₂=6.45±0.2, r₃=7.2±0.3 nm) decorated by surrounding Au Duff colloid of 2-5 nm radius; and (v) is the spectra for FeOx-Au—SiO₂—Au MSNPs (r₁=6, r₂=6.45±0.2, r₃=8.3±0.5, r₄=21±5 nm).

FIG. 4 is a series of plots showing: (A) the relaxometric response of FeOx-Au—SiO₂—Au MSNPs according to this disclosure at an absorbance of 0.1098 a.u. (T2 relaxation curve fit was conducted with a 95% confidence interval (n=4) of 2.72 ms); (B) that differences in the ratio between the size of the SiO₂ layer and the size of the outer Au layer for FeOx-Au—SiO₂—Au MSNPs causes a shift in the extinction shift in the NIR, where the top plot represents a first FeOx-Au—SiO₂—Au MSNP (r₁=6, r₂=6.45±0.2, r₃=8.3±0.5, r₄=21±5 nm) and the bottom plot represents a second FeOx-Au—SiO₂—Au MSNP (r₁=6, r₂=6.45±0.2, r₃=7.3±0.6, r₄=17±4 nm); and (C) the diameter histogram of a sample batch of FeOx-Au—SiO₂—Au MSNPs based on analysis of TEM images (average diameter was determined to be about 26.8±3.7 nm).

FIG. 5 is a plot showing the UV-Vis-NIR spectra of three different batches of FeOx-Ag nanoparticles.

FIG. 6 is a plot showing the UV-Vis-NIR spectra THPC-stabilized Ag nanoparticle precursors for decorating FeOx-Ag—SiO₂ nanoparticles with Ag.

FIG. 7 is a plot showing the UV-Vis-NIR spectra of four different batches of FeOx-Au—SiO₂ nanoparticles decorated with Ag using THPC-stabilized Ag nanoparticles.

### DETAILED DESCRIPTION

Optical, MRI, and CT based imaging contrast of pathologic tissues, therapeutic localization at the site of action at the cellular level, and the inability to unite diagnosis and treatment into a single entity continue to limit the practical power and application of these current emerging technologies. This disclosure provides multi-functional, multistrata nanoparticles (MSNPs) that have tunable dual-peak (Vis-NIR) extinction characteristics, tri-modal (optical, MRI and CT) imaging contrast, and small size (less than about 60 nm in diameter), and that are relatively easy to synthesize and/or to modify so as to include surface functional groups. This may provide for coupling diagnostics and therapeutics into a single theranostic material.

Multilayered nanoparticles (i.e., nanoparticles having a core with outer surrounding shells) may be classified as inorganic or hybrid organic-inorganic, and may be designed to provide new properties based on the characteristics of each individual layer in a synergistic fashion. Rational design principles can be employed to create nanomaterials with enhanced functional applications, such as tissue specific recognition, image contrast and therapeutic delivery. For example, FeOx-Au nanoparticles (i.e., nanoparticles having an iron-oxide core and a gold shell), have been synthesized to utilize both the magnetic relaxivity of the iron-oxide and surface plasmon resonance properties of the spherical gold shell. These nanoparticles have been implemented for simultaneous MR image contrast with cancer phototherapy. SiO₂—Au nanoparticles (i.e., nanoparticles having a silica core and a gold shell), have been clinically used for tissue-specific photothermal therapy optimized for in vivo use by design of the surface plasmonic properties of the nanomaterial. Unlike FeOx-Au nanoparticles, which have plasmonic extinction peaks in the visible spectrum, extinction peaks in the near-infrared (NIR, 700-1200 nm) can be achieved by control of the thickness ratio between the silica core and gold shell. Extinction peaks in the NIR allow for the optimal heating of subdermal tissue for photothermal therapy and efficient optical imaging. Harnessing the surface plasmon resonance properties of core/shell materials, the nanosphere-in-a-nanoshell (the “gold nanomatryushka”) was synthesized and demonstrated to provide specific extinction maxima in the UV-Vis-NIR spectrum that are associated with the nanoscale structure. A multilayered, metallodielectric nanostructure, the nanomatryushka includes a gold nanosphere surrounded by concentric silica/gold shells. Governed by surface plasmon hybridization theory, concentric metal layers separated by a dielectric spacer layer causes plasmon interactions which generate multi-peak extinction UV-Vis-NIR spectra. The location of the multi-extinction peaks are controlled by the metal shell and dielectric layer geometric ratio allowing for specific “tunability” of the optical characteristics of the nanostructure.

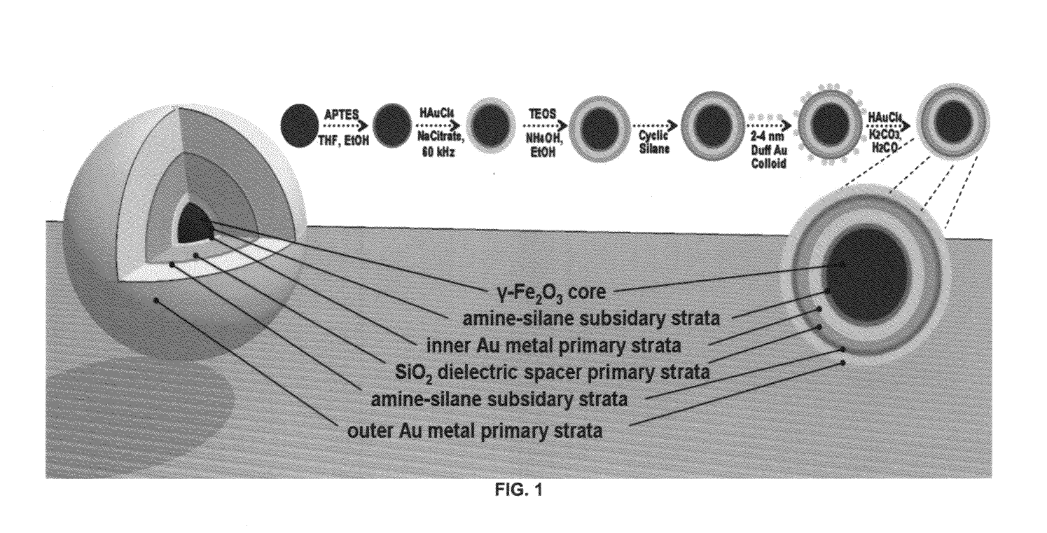

This disclosure provides multistrata nanoparticles (MSNPs) designed to exhibit MRI contrast, X-ray contrast for CT, photonic contrast for OCT, absorbance in the NIR for PTT, tunability of extinction characteristics during fabrication, theranostic potential, easy surface modulation for cellular targeting and biocompatibility. The MSNPs preferably have a nanostructure diameter of less than about 60 nm to support vascular extravasation ability. As discussed in more detail below, the MSNPs comprise a superparamagnetic iron oxide core (e.g., Fe₂O₃ or Fe₃O₄, etc.), a first shell formed of one or more plasmon active metals (e.g., gold, silver, copper, platinum, etc.) surrounding the core, a second shell formed of a dielectric material (e.g., SiO₂, among others) surrounding the first shell, and a third shell formed of one or more plasmon active metals (e.g., gold, silver, copper, platinum, etc.) surrounding the second shell. FIG. 1 shows an exemplary MSNP, and an exemplary method for fabricating the MSNP according to aspects of this disclosure. Specifically, FIG. 1 shows a FeOx-Au—SiO₂—Au MSNP (i.e., a MSNP where the plasmon active metal is Au). The MSNP resembles a single core, five layered “onion,” where each strata possesses a specific function.

In order to provide nanoparticles having so many functional layers, or strata, while still having such a small size, each strata must be carefully added through controlled fabrication methods. These methods permit the fabrication of extremely thin shells (as small as 1-2 nm to maintain an overall particle diameter less than about 100 nm, such as less than about 90 nm, less than about 80 nm, less than about 70 nm and preferably, less than about 60 nm) while still ensuring magnetic material retention throughout the fabrication process. Generally, the methods include coating a superparamagnetic iron oxide particle (e.g., Fe₂O₃ or Fe₃O₄, etc.) with a first aminosilane (e.g., APTES, APTMS, APDEMS, APEMS, etc.) to form an aminated core, coating the aminated core with a first shell formed of one or more plasmon active metals (e.g., gold, silver, copper, platinum, etc.), coating the first shell with a second shell formed of a dielectric material (e.g., SiO₂, among others) using sonication, coating the second shell with a second aminosilane (e.g., APTES, APTMS, APDEMS, or APEMS, a cyclic aminosilane such as N-n-butyl-aza-2,2-dimethoxysilacyclopentane, etc.) to form an aminated second shell, and coating the aminated second shell with a third shell formed of one or more plasmon active metals (e.g., gold, silver, copper, platinum, etc.). An exemplary method for fabricating the nanoparticle of FIG. 1 is shown in FIG. 1, and is further discussed in the Examples below. The Examples also discuss exemplary methods for making FeOx-Ag—SiO₂—Ag MSNPs.

The methods of this disclosure may include the preparation of superparamagnetic FeOx nanoparticle cores (e.g., Fe₂O₃ or Fe₃O₄, etc.). These methods are well known in the art, and may include, but are not limited to, coprecipitation of FeOx (e.g., by forming a suspension of Fe salts under basic conditions), microemulsion processes, and thermal decomposition of organic precursors (e.g., Fe(Cup)3, Fe(CO)₅, Fe(acac)3, etc.) in the presence of oxygen after aeration and reflux. FeOx nanoparticles also may be obtained commercially. The FeOx nanoparticles may have diameters between about 5 and about 95 nm, such as diameters less than about 85 nm, less than about 75 nm, less than about 65 nm, and preferably less than about 55 nm. In some cases, the FeOx nanoparticles may be synthesized using surfactants, such as oleic acid, to keep the particles from aggregating, and to provide FeOx nanoparticles having surface chemistry that enables subsequent chemical modification. In some cases, the surface of the FeOx nanoparticles may be functionalized using coatings having any of various functional groups.

The FeOx nanoparticles may be coated with a first aminosilane to form an aminated core (i.e., FeOx-NH₂). Suitable aminosilanes may include, but are not limited to APTES (i.e., (3-aminopropyl)triethoxysilane), APTMS, APDEMS, APEMS, etc. Many methods for coating FeOx nanoparticles with aminosilane are known, and are described in U.S. Pat. Nos. 4,628,037, 4,554,088, 4,672,040, 4,695,393 and 4,698,302, the complete teachings of which are herein incorporated by reference for all purposes. Conventional aminoxysilane reactions, such as those that utilize APTES, may involve single solvents such as DI H₂O, ethanol (EtOH), toluene and tetrahydrofuran (THF) and are fully detailed in synthetic chemical literature. For example, in cases, where the FeOx nanoparticle is coated with an oleic acid coating, the aminosilane may displace the oleic acid in an exchange reaction. One particular modification to the conventional APTES reaction may include the performance of multiple solvent exchanges throughout the reaction to optimize APTES deposition, —NH₂ availability, and magnetic material recovery. THF may be used as the primary solvent due to its ability to maximize APTES localization on the surface of the FeOx cores through both specific and non-specific bonding. The reaction solution may be spiked with a small amount of DI H₂O to catalyze the reaction and acetic acid to balance the reaction solution at pH˜6.5. The THF may be exchanged and washed with EtOH to release the non-specific APTES adsorption. When suspended in EtOH, the aminated FeOx cores may be highly colloidal and difficult to sediment by centrifugation; therefore, the washed cores may be added to hexanes to prepare the material for purification and extraction through centrifugation. Following three purification cycles, the FeOx-NH₂ particles may be suspended and stored in EtOH in preparation for the deposition of the first metal layer. Regardless of the method used to coat the FeOx with aminosilane, the aminosilane coating provides —NH₂ groups that have a high affinity for metals (e.g., Au, Ag, Cu, and Pt ions, among others) and may act as a coupling layer between the FeOx core and the initial plasmon active metal layer that is to be applied as a shell around the FeOx core.

Utilizing the high affinity between —NH₂ groups and metal ions, a thin primary strata formed of a plasmon active metal (PAM) may be added to the surface of the FeOx-NH₂ nanoparticle. The PAM may be added to the surface by any suitable method including, but not limited to, a sonochemical plating, or sonoplation, method. The sonoplation method employs the physiochemical effects of ultrasound which arise from acoustic cavitation (i.e., sonication). This effect can be physically described as the implosive collapse of bubbles formed at the surface of the FeOx-NH₂ nanoparticles. Through adiabatic compression, this collapse generates a localized hotspot due to the formation of a shockwave within the gas phase of the collapsing bubble. In their sonochemistry review, Mason and Lorimal described the empirically determined extreme, transient conditions of 5000 K temperatures, pressures of 1800 atm and cooling rates beyond 10¹⁰ K s⁻¹ at these hotspots (See Applied Sonochemistry. 2002, New York: Wiley). This extreme local environment formed by the sonoplation reaction produces similar conditions generated through conventional “high-heat, high-stir rate” nanoparticle and nanolayer formation methods implemented throughout nano-literature. Due to the inherent chelating ability between juxtaposed metal ions and available —NH₂ groups, a thin metal layer may be quickly deposited onto the surface of the FeOx-NH₂ nanoparticle to form an FeOx-PAM nanoparticle (e.g., an FeOx-Au nanoparticle, FeOx-Ag nanoparticle, FeOx-Cu nanoparticle, FeOx-Pt nanoparticle, etc.), such as through the use of sodium citrate as a reducing agent and the sonoplation ultrasonic frequency as a reaction catalyst.

A dielectric layer may be deposited onto the FeOx-PAM particle to form an FeOx-PAM-dielectric nanoparticle. Any suitable dielectric may be used, and may be added to the surface by any suitable method. For example, a sonoplation method may be used where tetraethyl orthosilicate (TEOS) is mixed with an alkaline initiator (NH₄OH) under ultrasonic agitation, thereby causing the deposition of a SiO₂ layer onto the PAM layer. The thickness of the SiO₂ may be carefully controlled by the ratio of FeOx-PAM particle volume to TEOS volume.

An intermediate aminosilane strata may be added to aminate the surface of the FeOx-PAM-dielectric layer in preparation for deposition of an additional PAM layer. In some cases, the aminosilane may be added according to the methods described above. In some cases, a cyclic aminosilane, such as N-n-butyl-aza-2,2-dimethoxysilacyclopentane, may be coated onto the dielectric layer to avoid the multi-step process required by APTES amination and to reduce the possibility of particle flocculation due to the generation of reaction side products and self-polymerization.

Following the silanization of the surface of the FeOx-PAM-dielectric particle (i.e., to form an FeOx-PAM-dielectric-NH₂ nanoparticle), the available —NH₂ sites may be used to deposit a final PAM strata (i.e., to form to an FeOx-PAM-dielectric-PAM MSNP). Various methods may be used to deposit the final PAM layer, depending on the desired thickness of the layer, the rate at which deposition is desired, etc. In some embodiments, the final PAM layer may be deposited by first decorating the FeOx-PAM-dielectric-NH₂ nanoparticle with metal colloids, which may act as nucleation or “seed” sites for subsequent metal deposition through the reduction of metal ions in the presence of a reducing agent. For example, the available —NH₂ sites of the FeOx-PAM-dielectric-NH₂ nanoparticles may be decorated with Duff Au colloids (e.g., 2-5 nm Duff Au colloids), and then a complete Au layer may be catalyzed onto the decorated particles through the reduction of a HAuCl₄ solution in the presence of H₂CO (i.e., a formaldehyde electroless plating reaction). Alternatively or additionally, the —NH₂ sites may be decorated with THPC-stabilized metal colloids (e.g., THPC-stabilized Ag) prior to depositing the final PAM layer.

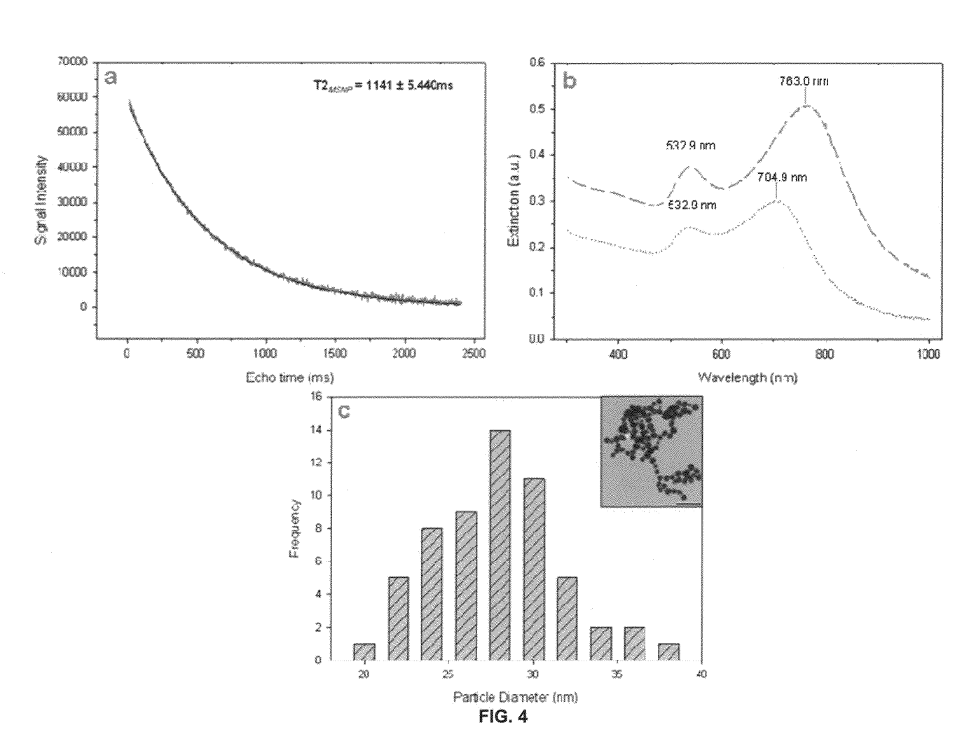

In order to maintain the stability of the outer PAM strata, the MSNPs may be resuspended in a solution containing a stabilizing agent. For example, when an FeOx-Au—SiO₂—Au MSNP was resuspended in a 1.8 mM solution of K₂CO₃, a zeta potential of −75.6±0.902 was measured, which is consistent with the existence of —CO₃²⁻ ions stabilizing the surface of the particle. With respect to MSNPs having PAM layers comprising gold, which present the same surface chemistry, these stabilizing ions are easily place-exchanged with a number of conjugates that proximally present amine, sulfhydryl or other functional groups. From a surface modification perspective, MSNP behavior is the same as for other particles, for which many robust methods are well known to provide a wide range of molecular coatings and/or functional groups. These fabrication reactions yield monodisperse batches of nanoparticles, as shown in FIG. 4(C). In addition, these reactions are scaleable, making the fabrication of bulk quantities possible.

For more than half a decade, PAM-coated nanoparticles, such as gold-coated nanoparticles, have been shown to act as X-ray and CT contrast agents, increasing the utility of each technique imaging biological samples. MSNP capacity for MRI contrast can be evaluated through relaxometric measurements. As discussed in the Examples below, the extinction peak of FeOx-Au—SiO₂—Au MSNPs located in the visible spectrum was measured at 0.1098 a.u. The MSNPs exhibited a relaxation time at 1141±5.4 ms fit via a 95% confidence interval based on four repetitions. This relaxation time can be differentiated from the T2 values of healthy human tissue, and are predicted to alter the relaxation times of proximal tissues. These results support the MR contrast capacity of FeOx-PAM-dielectric-PAM MSNPs, such as FeOx-Au—SiO₂—Au MSNPs, among others.

This disclosure provides the fabrication of a single core, five layered nanostructure with the potential capacity for both CT and MR imaging contrast. This contrast agent may allow for the simultaneous use of both technologies and as well as other hybrid imaging modalities. As described in more detail below, these particles have been characterized to show their metallodielectric properties and dual-peak UV-Vis-NIR extinction spectra. This disclosure also provides evidence of the geometric-dependent ‘tunability’ of the optical extinction characteristics of the MSNPs, which may allow predictable optimization of performance based on controllable synthetic conditions. This technology may be further applied for laser absorption and consequent thermal characteristics in the NIR. Successful demonstration of significant optical absorption and heat generation is consistent with future applications in theranostic disease treatment.

The methods and apparatus disclosure herein are not limited in their applications to the details of construction and the arrangement of components described herein. The invention is capable of other embodiments and of being practiced or of being carried out in various ways. Also it is to be understood that the phraseology and terminology used herein is for the purpose of description only, and should not be regarded as limiting. Ordinal indicators, such as first, second, and third, as used in the description and the claims to refer to various structures, are not meant to be construed to indicate any specific structures, or any particular order or configuration to such structures. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification, and no structures shown in the drawings, should be construed as indicating that any non-claimed element is essential to the practice of the invention.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

Further, no admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinency of any of the documents cited herein.

### EXAMPLES

### Example 1—Particle Characterization

The MSNPs and the various precursor nanoparticles described in these Examples were characterized using transmission electron microscopy (TEM) with a Phillips CM20 microscope, spectroscopy using a Varian Cary 50 UV-Vis-NIR spectrophotometer, and/or relaxometry using a Maran DRX-II 0.5T NMR spectroscopic scanner following sample preparation using 5 ml of 1×PBS as a solvent. Zeta potential measurements were obtained using a Malvern Zetasizer (Malvern Instruments, Westborough, Mass.) following sample preparation using 1 ml of 1.8 mM K₂CO₃ as a solvent. Particle sizes and statistical distributions were estimated from TEM images using Amt V600 and ImageJ software.

### Example 2—Fabrication of FeOx Nanoparticle Cores

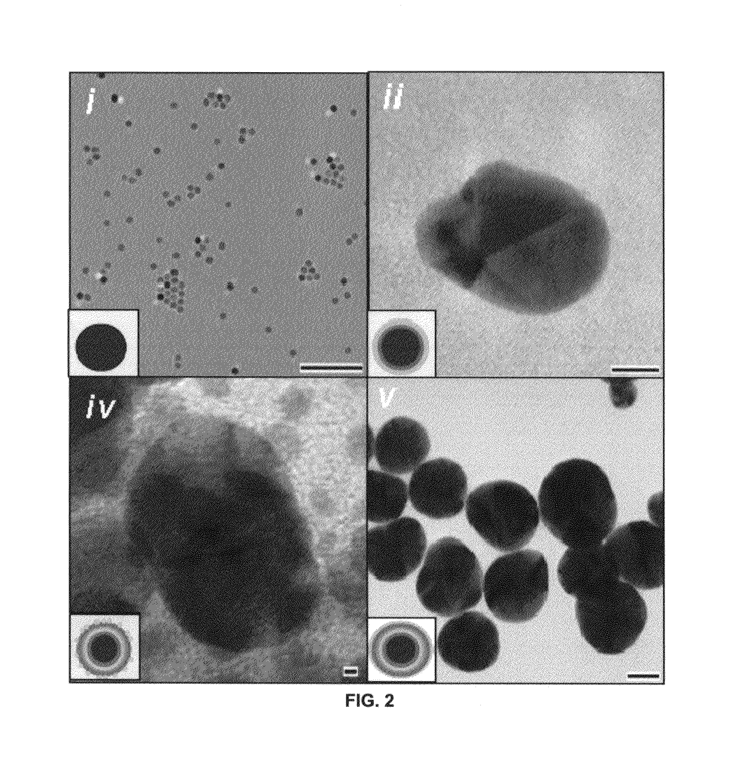

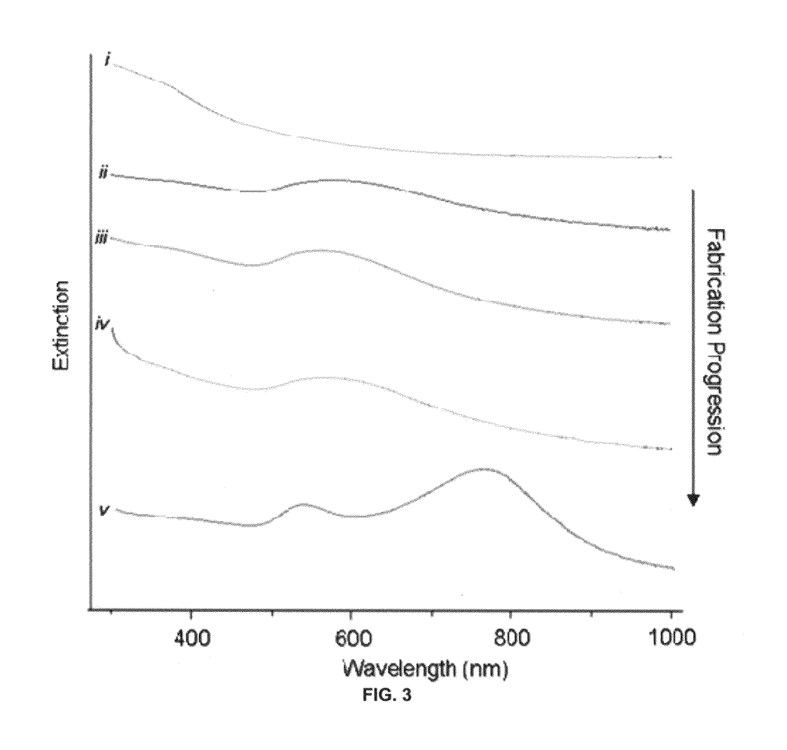

γ-Fe₂O₃ particles with 12±1 nm diameters were fabricated by a thermal decomposition, aeration and reflux protocol. Briefly, 20 ml of octyl ether (Sigma-Aldrich, St. Louis, Mo.) and 1.92 ml of oleic acid (Sigma-Aldrich) were stirred under N₂ gas flow and reflux. The sample was heated to 100° C. prior to addition of 0.4 ml Fe(CO)₅ (Sigma-Aldrich). The reaction was heated from 150° C. to 280° C. where the reaction solution color changed from boil, to orange, orange/colorless, to very dark orange. Sample was aerated at 80° C. for 14 hours and refluxed while boiling for 2 hours. The γ-Fe₂O₃ cores were centrifuged (15 min, 770 rcf) and washed in ethanol (EtOH, 200 proof, Sigma-Aldrich) twice, and dried under air. FIG. 2(i) shows the transmission electron microscopy (TEM) image of the FeOx nanoparticles, and FIG. 3(i) shows the UV-Vis-NIR Spectra of the FeOx nanoparticles.

### Example 3—Amination of FeOx Nanoparticles to Form FeOx-NH₂

150 mg of γ-Fe₂O₃ core (FeOx) were coated with the first strata using a modified (3-aminopropyl)triethoxysilane (APTES, Sigma-Aldrich) functionalization. Core particles were added to 40 ml of tetrahydrofluran (THF, Thermo Fisher Scientific, Waltham, Mass.) and stirred briskly using a magnetic stir plate and stirring rod. 5 ml of APTES was added to the reaction solution and thereafter spiked with 5 μl of acetic acid (Sigma-Aldrich), 516 μl of MilliQ (18 MΩ) DI H₂O, and stirred for 48 hours. The reaction flask was then placed in a glycerol bath and heated to 80° C. EtOH was used to replace evaporated THF throughout the 2 hour boiling period. Reaction solution was concentrated via rotovap to 40 ml of EtOH. Hexane was added to the solution in a 4:1 ratio and centrifuged (10 min, 800 rcf). Recovered FeOx-NH₂ nanoparticles were resuspended in EtOH and stored at room temperature where they remained stable throughout the length of this study (>5 months).

### Example 4—Gold Plating of FeOx-NH₂ Nanoparticles to Form FeOx-Au Nanoparticles

FeOx-NH₂ nanoparticles were coated with a gold layer. Sonicated FeOx-NH₂ nanoparticles (˜1.5% wt) were added in equal volume to DI H₂O-based, 1% HAuCl₄ (Sigma-Aldrich, dark aged 24-72 hours) under ultrasonic perturbation. 20 mM sodium citrate in DI H₂O was added dropwise under sonication. A distinct color change from a flocculated (due to immediate repulsive electrostatic interactions prior to Au³⁺ liberation by the sodium citrate) yellow-brown mixture to a black-purple, fully colloidal suspension, following the induction of the reducing agent and catalyst, signaled the generation of the FeOx-Au nanoparticles. The solution was then washed via centrifugation (5 min, 800 rfc) and resuspended in EtOH and stored for 18 hours at 4° C. where the FeOx-Au nanoparticles remained stable for 5 months.

FIG. 2(ii) shows the transmission electron microscopy (TEM) image of the FeOx-Au nanoparticles, and FIG. 3(ii) shows the UV-Vis-NIR Spectra of the FeOx-Au nanoparticles. The addition of the gold shell around the FeOx core was substantiated by the appearance of a surface plasmon resonance extinction maxima (λₘₐₓ540-570 nm) in the sample absorbance spectra for the FeOx-Au particle (FIG. 3(ii)) which is not evident in the FeOx nanoparticles (FIG. 3(i)). In addition, a comparison of the high-resolution TEM images of the FeOx nanoparticles (FIG. 2(i)) and FeOx-Au nanoparticles (FIG. 2(ii)) clearly shows the formation of gold “plates” on the iron oxide surface and the development of gold fringe patterns (111 planes, 0.24 nm) consistent with well characterized images in electron microscopy literature.

### Example 5—Formation of FeOx-Au—SiO₂ Nanoparticles

FeOx-Au nanoparticles were coated with a silica layer. 1 ml of a solution of FeOx-Au nanoparticles in EtOH from Example 4 was added to 5 ml of fresh EtOH. Under ultrasonic perturbation, 35 μl of 0.4% NH₄OH and 25-50 μl of 10 mM ethanolic tetraethyl orthosilicate (TEOS, Sigma-Aldrich) were added. Sonication was continued at room temperature for 45 minutes and thereafter stored at 4° C. for 24 hours to form the FeOx-Au—SiO₂. FIG. 3(iii) shows the UV-Vis-NIR Spectra of the FeOx-Au—SiO₂ nanoparticles. The thickness of the dielectric layer may be modulated by varying the relative amount of TEOS and FeOx-Au nanoparticles in the reaction mixture. As discussed in more detail below, modulation of the thickness of the dielectric layer affects the spectral properties of the MSNPs made according to the present disclosure.

### Example 6—Formation of FeOx-Au—SiO₂-NH₂ Nanoparticles

The FeOx-Au—SiO₂ nanoparticles were coated with N-n-butyl-aza-dimethoxysilacyclopentane (cyclic silane, Gelest, SIB1932.4). 400 μl of 1 mM ethanolic cyclic silane was added under ultrasonic perturbation to the ethanolic suspension of FeOx-Au—SiO₂ nanoparticles to form FeOx-Au—SiO₂—NH₂ nanoparticles. The solution of FeOx-Au—SiO₂—NH₂ nanoparticles was stored at 4° C. for 24 hours where they remained stable until completely utilized (>3 months).

### Example 7—Formation of FeOx-Au—SiO₂—Au MSNPs

NH₂—SiO₂—Au-FeOx nanoparticles were decorated through emersion in Duff gold colloid (2-4 nm, dark-aged for 3 weeks in 4° C.) in a 1:4, particle to colloid ratio. Briefly, 1 ml of NH₂—SiO₂Au-FeOx was mixed with 4 ml of Au Duff colloid. This mixture was left unperturbed at room temperature (20-23° C.) for 24 to 96 hours, centrifuged (10 min, 800 rcf), supernatant removed via magnetic assisted aspiration and resuspended in 1 ml of MilliQ DI H₂O via ultrasonic sonication. More specifically, magnetic assisted aspiration is conducted via a 1 Tesla neodymium 1″ cube magnet (CMS Magnetics, Plano, Tex.) placed at the bottom of the reaction vial in order to retain magnetic material in its pellet form during aspiration. These decorated particles were immediately used for the next step. FIG. 2(iv) shows the transmission electron microscopy (TEM) image of the FeOx-Au—SiO₂ nanoparticles decorated by surrounding Au Duff colloid of 2-5 nm radius, and FIG. 3(iv) shows the UV-Vis-NIR Spectra of the FeOx-Au—SiO₂ nanoparticles decorated by surrounding Au Duff colloid.

Decorated particles were vigorously mixed with a 1% HAuCl₄-K₂CO₃ plating solution in a 1:10 ratio. Briefly, 25 mg of K₂CO₃ (Sigma-Aldrich) was added to 100 ml of H₂O where 1% HAuCl₄ (dark-aged for 14 days prior) was added and dark-aged for 96 hours. 10 μl of H₂CO (Sigma-Aldrich) was added as a catalyst which began the release of Au ions thus causing a color change from clear to bright pink. Following a 10 min reaction time, particles were centrifuged (10 min, 800 rcf) and the supernatant was removed via magnetic assisted aspiration. Completed MSNPs were re-suspended in 1 ml of EtOH, thus quenching the plating solution, and stored at 4° C. for further characterization. For storage longer than 10 days, MSNPs were re-suspended in 1 ml of 1.8 mM K₂CO₃ at 4° C. The deposition of this final PAM layer is supported by the formation of a metallic outer layer and a change in surface plasmon extinction spectra (see FIGS. 2(v) and 3(v)). For example, a double-peak spectra of a multilayered, gold-dielectric-gold material appeared following electroless plating (FIG. 3(v)).

As shown in FIG. 4(A), the relaxometric response of FeOx-Au—SiO₂—Au MSNPs at an absorbance of 0.1098 a.u. was determined, and the MSNPs were found to exhibit a relaxation time at 1141±5.4 ms fit via a 95% confidence interval based on four repetitions. This relaxation time can be differentiated from the T2 values of healthy human tissue, and are predicted to alter the relaxation times of proximal tissues. These results support the MR contrast capacity of FeOx-Au—SiO₂—Au MSNPs.

As shown in FIG. 4(B), modulation of the thickness of the dielectric layer (as discussed in Example 5 above) affected the spectral properties of the MSNPs made according to the present disclosure. The thickness was modulated by varying the relative amount of TEOS and FeOx-Au nanoparticles when forming the FeOx-Au—SiO₂ nanoparticles. Extinction maxima shifted from λ₁=533 nm and λ₂=705 nm to λ₁=533 nm and λ₂=763 nm when an additional 25 μl of TEOS was provided.

The fabrication reactions described herein yield monodisperse batches of FeOx-Au—SiO₂—Au MSNPs, as shown in FIG. 4(C).

### Example 8—Silver Plating of FeOx-NH₂ Nanoparticles to Form FeOx-Ag Nanoparticles

Reaction mixtures were formed by mixing 500 μl of a 1% by weight FeOx-NH₂ nanoparticle solution to 40 μl of sodium citrate solution and solutions containing 0.431% AgNO₃ and (reaction mixture 1s=1.75 ml AgNO₃ solution, 2s=2.00 AgNO₃ solution, and 3s=AgNO₃ solution). The reaction mixtures were allowed to sonicate for 5 minutes, after which 5 μl of a 0.04% NaOH solution was added. 40 μl of sodium citrate was subsequently added in 30 second increments (with slight manual swirling every 5^{th} addition) until 1.6 ml of sodium citrate total was added (or 20 minutes of sonication time). The reaction was left unperturbed for 1 hour, and a distinct color change from a yellow-brown to peach hue was observed.

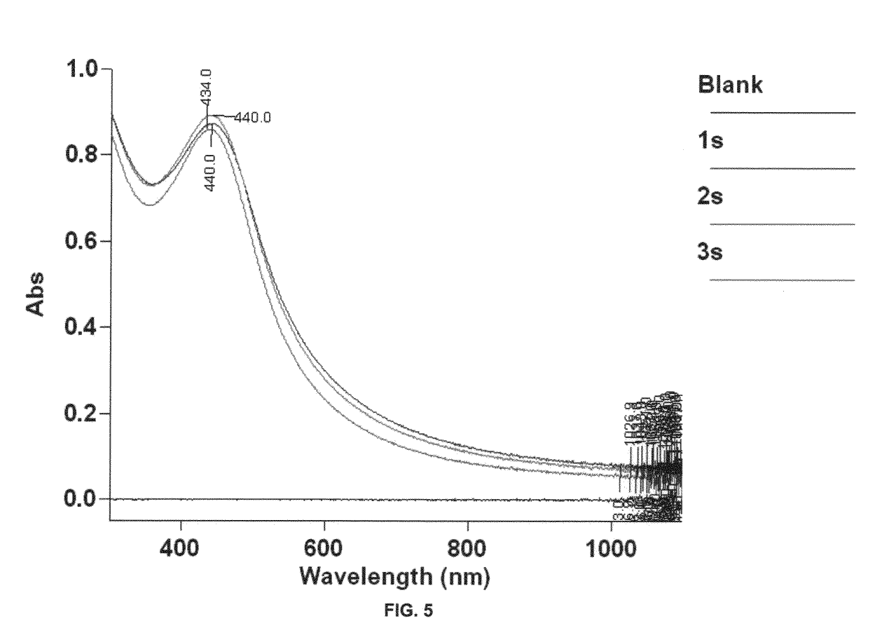

FIG. 5 is a plot showing the UV-Vis-NIR spectra of the products of reaction mixtures 1s, 2s and 3s, which shows peaks at about 440 nm corresponding to the presence of silver.

### Example 8—Formation of FeOx-Ag—SiO₂and FeOx-Ag—SiO₂—NH₂ Nanoparticles

The silica and cyclic-silane layers were added to the FeOx-Ag nanoparticles following the same protocol described above with respect to the addition of the equivalent layers to the FeOx-Au nanoparticles.

### Example 9—Formation of FeOx-Ag—SiO₂—Au MSNPs

THPC-stabilized Ag nanoparticles were synthesized by adding 1.2 ml of 1M NaOH to 180 ml of MilliQ H₂O in a 250 ml beaker. A small stir bar was added and the reaction mixture was allowed to stir at 50% max rate for 5 minutes. 4 ml of 0.95% THPC (Tetrakis(hydroxymethyl)phosphonium chloride) solution was added, and the reaction mixture was allowed to continue stirring for 5 minutes. The entire reaction solution was then added to a fresh reaction vessel, and 69 μl of 0.04% NH₄OH was added, and 6.75 ml of AgNO₃ (0.431% soln) was added under 100% vortex level. The color changed from clear to a brown, dark brown “cola” color.

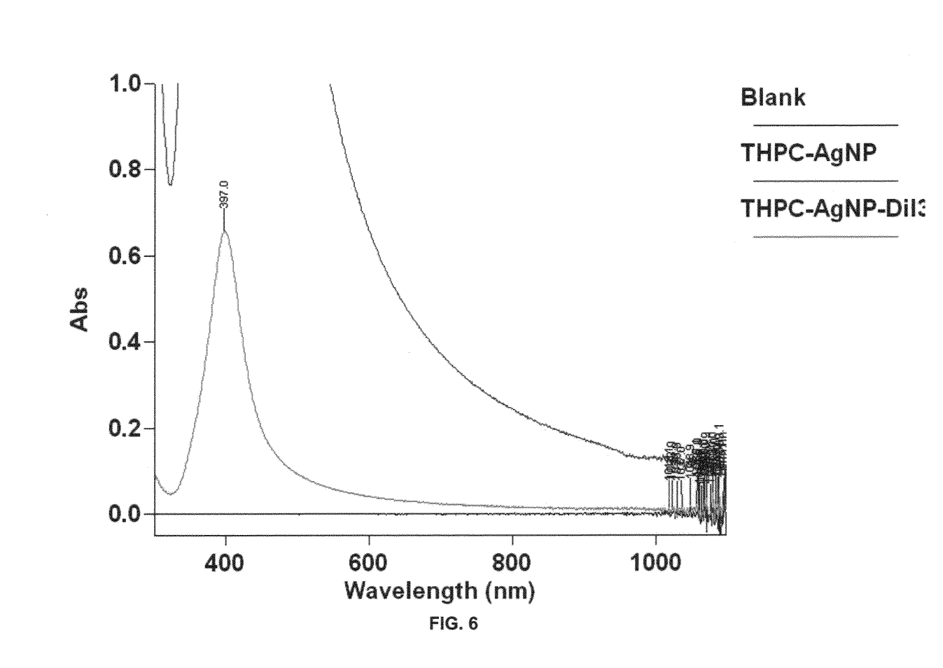

FIG. 6 is a plot showing the UV-Vis-NIR spectra of the THPC-stabilized Ag nanoparticle precursors for decorating FeOx-Ag—SiO₂ nanoparticles with Ag. The plot shows the extinction peaks at 397.0 nm (i.e., a characteristic silver peak).

400 μl of THPC-Ag nanoparticles were added to 100 μl of FeOx-Ag—SiO₂—NH₂ nanoparticles. Upon mixing, an immediately color change occurred, causing the solution to turn dark amber red, and then after approximately 45-60 seconds, redish purple, supporting the conclusion that FeOx-Au—SiO₂ nanoparticles were decorated with the Ag particles.

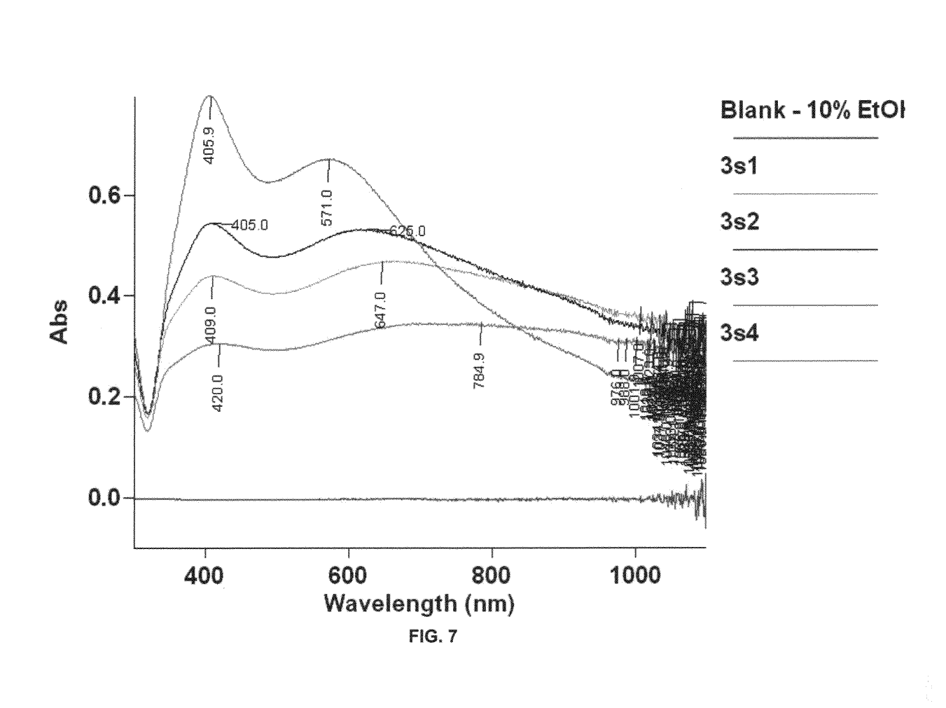

FIG. 7 is a plot showing the UV-Vis-NIR spectra of four different batches of FeOx-Au—SiO₂ nanoparticles decorated with Ag using THPC-stabilized Ag nanoparticles. As with the FeOx-Au—SiO₂—Au MSNPs discussed above, dual peak spectra were observed with primary peaks showing the presence of silver and secondary peaks providing evidence of plasmon resonance hybridization. The four samples shown in FIG. 7 differ in that they have varying silica strata thicknesses (thus causing the variations in the secondary peaks).

After adding the FeOx-Ag—SiO₂ nanoparticles are seeded with Ag, the final Ag layer may be plated in a similar manner to the final gold layer in the FeOx-Au—SiO₂—Au MSNPs, and preferably to a thickness of <3 nm of silver to maintain plasmonic interactions.

It should be appreciated that the fundamental difference between Au and Ag containing MSNPs is their plasmon active metal. Varying the metals may provide MSNPs having different applications. For example, silver particles generally have secondary peaks in the 500-700 nm “visible” spectrum, thus eliminating their potential for use in photothermal therapy, via NIR laser irradiation, due to the fact that they generally do not highly absorb at NIR wavelengths. In contrast, gold particles are generally NIR sensitive and may be used in photothermal therapeutic procedures. As shown above, silver MSNPs can be designed to absorb in the NIR, however their absorbance in this spectra range is relatively nominal as compared to their gold counterparts. Silver has documented bactericidal properties which could be leveraged in a number of external medical applications. In vivo uses of silver are not widely studied as there are fears of toxicity in humans and laboratory animals. Both of these particles could be utilized in a number of laboratory-based or clinical (AuMSNPs) imaging applications. New (cutting-edge) hybrid imaging modalities which depend on the combined biomagnetophotonic properties of their imaging contrast agents could employ both AuMSNPs and AgMSNPS as potential contrast agents.

### REFERENCES

The following references are herein incorporated by reference in their entireties for all purposes:
[1] R. Popovtzer, A. Agrawal, N. Kotov, A. Popovtzer, J. Baiter, T. Carey, R. Kopelman, Targeted Gold Nanoparticles enable Molecular CT Imaging of Cancer. Nano Lett., 2008: 8(12): p. 4593-4596.
[2] M. Saksena, M. Harisinghani, P. Hahn, J. Kim, A. Saokar, B. King and R. Weissleder Comparison of Lymphotropic Nanoparticle-Enhanced MRI Sequences in Patients with Various Primary Cancers. American Journal of Roentgenology, 2006. 187: p. 582-588.
[3] O. Will, S. Purkayastha, C. Chan, T. Athanasiou, A. Darzi, W. Gedroyc and P. P Tekkis, Diagnostic precision of nanoparticle-enhanced MRI for lymph-node metastases: a meta-analysis The Lancet Oncology, 2006. 7(1): p. 52-60.
[4] T. Islam, M. Harisinghani, Overview of nanoparticle use in cancer imaging Cancer Biomarkers, 2009. 5(2): p. 61-67.
[5] M. Yezhelyev, X. Gao, Y. Xing, A. Al-Hajj, S. Nie, R. O'Regan, Emerging use of nanoparticles in diagnosis and treatment of breast cancer. The Lancet Oncology, 2006. 7: p. 657-667.
[6] Runge, V., Contrast media research. Invest. Radiol., 1999. 34: p. 785-790.
[7] L. E. Ginsberg, G. Fuller, M. Hashmi, N. Leeds and D. Schomer, The Significance of Lack of MR Contrast Enhancement of Supratentorial Brain Tumors in Adults: Histopathological Evaluation of a Series Surgical Neurology, 1998. 49(9): p. 436-440.

[8] G. Antoch, L. Freudenberg, T. Egelhof, J. Stattaus, W. Jentzen, J. Debatin and A. Bockisch, Focal Tracer Uptake: A Potential Artifact in Contrast-Enhanced Dual-Modality PET/CT Scans The Journal of Nuclear Medicine, 2002. 43(10): p. 1339.
[9] N. Portney, M. Ozkan, Nano-oncology: drug delivery, imaging, and sensing Analytical and Bioanalytical Chemistry, 2006. 384(3): p. 620-630.
[10] L. Johnson, A. Gunasekeram, M. Douek, Applications of Nanotechnology in Cancer. Discovery Medicine, 2010. 9(47): p. 374-379.
[11] Schärtl, W, Current directions in core-shell nanoparticle design. Nanoscale, 2010. 2: p. 829-843.
[12] L. Hirsch, A. Gobin, A. Lowery, F. Tam, R. Drezek, N. Halas and J. West, Metal Nanoshells Annals of Biomedical Engineering, 2006. 34(1): p. 15-22.
[13] R. Hao, R. Xing, Z. Xu, Y. Hou, S. Gao, S. Sun, Synthesis, functionalization, and biomedical applications of multifunctional magnetic nanoparticles. Advanced Materials, 2010. 22(25): p. 2729-2742.
[14] M Melancon, W Lu, C Li, Gold-Based Magneto/Optical Nanostructures: Challenges for In Vivo Applications in Cancer Diagnostics and Therapy. Mater Res Bull, 2009. 34(6): p. 415-421.
[15] J. Aaron, J. Oh, T. Larson, S. Kumar, T. Milner, K. Sokolov, Increased optical contrast in imaging of epidermal growth factor receptor using magnetically actuated hybird gold/iron oxide nanoparticles. Optics Express, 2006. 14(26): p. 12930-12943.
[16] C. Levin, C. Hofmann, T. Ali, A. Kelly, E. Morosan, P. Nordlander, K. Whitmire and N. Halas, Magnetic-Plasmonic Core-Shell Nanoparticles. ACS Nano, 2009. 3(6): p. 1379-1388.
[17] L. Wang, H Y. Park, S. Lim, M. Schadt, D. Mott, J. Luo, X. Wang and C J. Zhong, Core@shell nanomaterials: gold-coated magnetic oxide nanoparticles. J. Mater. Chem., 2008. 18: p. 2629-2635.
[18] D. Kirui, D. Rey, C. Batt, Gold hybrid nanoparticles for targeted phototherapy and cancer imaging Nanotechnology, 2010. 21(10).
[19] L. Hirsch, R. Stafford, J. Bankson, S. Sershen, B. Rivera, R. Price, J. Hazle, N. J. Halas, J. L. West Nanoshell-mediated near-infrared thermal therapy of tumors under magnetic resonance guidance. PNAS, 2003. 100(23): p. 13549-13554.
[20] R. Bardhan, S. Mukherjee, N. A. Mirin, S. Levit, P. Nordlander, N. J. Halas, Nanosphere-in-a-Nanoshell: A Simple Nanomatryushka. J. Phys. Chem. C, 2010. 114(16): p. 7378-7383.
[21] Halas, C. Radloff N. J., Plasmonic Properties of Concentric Nanoshells. Nano Lett., 2004. 4(7): p. 1323-1327.
[22] K. Woo, J. Hong, S. Choi, H. W. Lee, J. P. Ahn, C. S. Kim, and S. W. Lee, Easy Synthesis and Magnetic Properties of Iron Oxide Nanoparticles. Chem. Mater., 2004. 16(14): p. 2814-2818.
[23] I. Bruce, T. Sen, Surface Modification of Magnetic Nanoparticles with Alkoxysilanes and Their Application in Magnetic Bioseparations. Langmuir, 2005. 21(15): p. 7029-7035.
[24] T. Mason, J. Lorimal, Applied Sonochemistry. 2002, New York: Wiley.
[25] W. Wu, Q. He, H. Chen, J. Tang and L. Nie, Sonochemical synthesis, structure and magnetic properties of air-stable Fe3O4/Au nanoparticles Nanotechnology, 2007. 18(14).

[26] M. Yamamoto, Y. Kashiwagi, T. Sakata, H. Mori, and M. Nakamoto, Synthesis and Morphology of Star-Shaped Gold Nanoplates Protected by Poly(N-vinyl-2-pyrrolidone). Chem. Mater., 2005. 17(22): p. 5391-5393.
[27] D. Duff, A. Baiker, P. Edwards, A new hydrosol of gold clusters, 1: formation and particle size variation. Langmuir, 1993. 9(2301-2309).
[28] B. Brinson, J. Lassiter, C. Levin, R. Bardhan, N. Mirin and N J. Halas, Nanoshells Made Easy: Improving Au Layer Growth on Nanoparticle Surfaces. Langmuir, 2008. 24(24): p. 14166-14171.
[29] J. Hainfeld, D. Slatkin, T. Focella, H. Smilowitz, Gold nanoparticles: a new X-ray contrast agent. The British Journal of Radiology, 2006. 79: p. The British Journal of Radiology.
[30] M W Groch, J A Urbon, W D Erwin, and S Al-Doohan, An MRI Tissue Equivalent Lesion Phantom Using A Novel Polysaccharide Material. Magnetic Resonance Imaging, 1991.9: p. 417-421.
[31] L. Hirsch, N J. Halas, J L. West, Whole-Blood Immunoassay Facilitated by Gold-Nanoshell-Conjugate Antibodies, in NanoBiotechnology Protocols, D. W. S J. Rosenthal, Editor. 2005, Humana Press: Totowa, N.J. p. 101-111.
[32] U.S. Pat. No. 4,554,088
[33] U.S. Pat. No. 4,628,037
[34] U.S. Pat. No. 4,672,040
[35] U.S. Pat. No. 4,695,393
[36] U.S. Pat. No. 4,698,302

We claim:

1. A composition comprising:
a core comprising iron oxide;
a first shell comprising at least one plasmon active metal at least partially surrounding the core;
a second shell comprising a dielectric material at least partially surrounding the first shell; and
a third shell comprising at least one plasmon active metal at least partially surrounding the second shell.

2. The composition of claim 1, wherein the composition has a diameter less than about 60 nm.

3. The composition of claim 1, wherein the iron oxide comprises superparamagnetic iron oxide.

4. The composition of claim 3, wherein the superparamagnetic iron oxide comprises at least one of Fe₂O₃, Fe₃O₄, and a combination thereof.

5. The composition of claim 1, wherein the first shell comprises at least one of gold, silver, copper, platinum, and a combination thereof.

6. The composition of claim 1, wherein the dielectric material comprises SiO₂.

7. The composition of claim 1, wherein the third shell comprises at least one of gold, silver, copper, platinum, and a combination thereof.

8. A method of making a nanoparticle, comprising:
forming a first shell at least partially surrounding a particle comprising iron oxide, the first shell comprising at least one plasmon active metal;
forming a second shell at least partially surrounding the first shell, the second shell comprising a dielectric material;
forming a third shell at least partially surrounding the second shell, the third shell comprising at least one plasmon active metal.

9. The method of claim 8, wherein the iron oxide comprises superparamagnetic iron oxide.

10. The method of claim 9, wherein the superparamagnetic iron oxide comprises at least one of Fe₂O₃ and Fe₃O₄.

11. The method of claim 8, wherein the first shell comprises at least one of gold, silver, copper, and platinum.

12. The method of claim 8, wherein the step of forming the first shell comprises coating the particle with an aminosilane to form an aminated core, and coating the aminated core with the first shell.

13. The method of claim 12, wherein the aminosilane comprises at least one of APTES, APTMS, APDEMS, and APEMS.

14. The method of claim 8, wherein the dielectric material comprises SiO₂.

15. The method of claim 8, wherein the step of forming the second shell comprises coating the first shell with the dielectric material using sonication.

16. The method of claim 8, wherein the third shell comprises at least one of gold, silver, copper, and platinum.

17. The method of claim 8, wherein the third shell has an exterior surface with a diameter less than about 60 nm.

18. The method of claim 8, wherein the step of forming the third shell comprises coating the second shell with an aminosilane to form an aminated second shell, and coating the aminated second shell with the third shell.

19. The method of claim 18, wherein the aminosilane comprises at least one of APTES, APTMS, APDEMS, APEMS and a cyclic aminosilane.

20. The method of claim 19, wherein the second aminosilane comprises a cyclic aminosilane.

21. The method of claim 20, wherein the cyclic aminosilane comprises N-n-butyl-aza-2,2-dimethoxysilacyclopentane.
